(19) United States
(12) Patent Application Publication
(10) Pub. No.: US 2015/0004300 A1
(43) Pub. Date: Jan. 1, 2015

(54) GLUTEN-FREE FOOD COMPOSITION AND FOOD PRODUCTS PREPARED THEREFROM

(71) Applicant: ATIS S.R.L., Milano (IT)

(72) Inventor: Alessandro Guido Cavalieri Manasse, Copparo (IT)

(21) Appl. No.: 14/350,154

(22) PCT Filed: Oct. 4, 2012
(86) PCT No.: PCT/IB2012/055331

§ 371 Date: Apr. 7, 2014

(30) Foreign Application Priority Data
Oct. 5, 2011 (IT) MI2011A001808

(51) Int. Cl. A21D 13/06; A23L 1/16; A23G 1/48

(52) U.S. Cl. CPC A21D 13/066; A23G 1/48; A23L 1/16; A23V 2002/00
USPC 426/549; 426/615; 426/622

(57) ABSTRACT

A gluten-free food composition consisting of at least one derivative of Cannabis Sativa, at least one derivative of 5-methyltetrahydrofolic acid and suitable food ingredients is described. In particular, products prepared with this food composition are perfectly suitable for the diet of gluten-intolerant patients, allowing not only the absorption of substances not tolerated by the bodies of such patients to be avoided, but also allowing their diet to be supplemented with elements that are useful in re-establishing the correct functioning of the body.

### FIELD OF THE INVENTION

The present invention relates to a gluten-free food composition consisting of at least one derivative of Cannabis Sativa, at least one derivative of 5-methyltetrahydrofolic acid and suitable food ingredients.

In particular, products prepared with this food composition are perfectly suitable for the diet of gluten-intolerant patients, allowing not only the absorption of substances not tolerated by the bodies of such patients to be avoided, but also to allowing their diet to be supplemented with elements that are useful in re-establishing the correct functioning of the body.

### BACKGROUND OF THE INVENTION

Coeliac disease is not a simple food intolerance but a true autoimmune disease. The immune reaction is triggered by gluten, a protein complex that is present in is certain cereals, which, due to some of its fractions, known as prolamins, generates the toxic effect in coeliac patients. In wheat, Prolamin is called gliadin, while other similar proteins that have the same effect on coeliacs, are found in barley, rye, emmer, spelt, khorasan wheat and triticale.

When a coeliac ingests gluten through food, the immune system reacts by attacking the inner wall of the intestine, causing lesions and atrophy of the villi, structures responsible for the absorption of all the nutrients necessary for the growth and maintenance of our body. The flattening of the intestinal villi and the consequent malabsorption of nutrients are responsible for many of the symptoms of coeliac disease and contribute to the onset of certain diseases such as anaemia and osteoporosis.

Coeliac disease can present a wide spectrum of clinical manifestations, ranging from signs and symptoms of frank malabsorption to more insidious and muted pictures. In the presence of the typical symptoms of severe malabsorption (diarrhoea, steatorrhoea and marked weight loss), it is known as “major coeliac disease”.

In the case of minor and extra-intestinal symptoms (anaemia, osteoporosis, typical skin lesions, infertility, spontaneous abortions, etc.), it is known as “minor coeliac disease”.

Lastly, in those patients who are diagnosed but present no symptoms, usually running in families, it is known as “silent coeliac disease”

The reversibility of the disease is closely connected to the non-ingestion by coeliac subjects of food containing gluten or even merely contaminated therewith.

However, there is no cure for coeliac disease, thus determining the need for coeliac subjects to adopt a strictly gluten-free diet for life.

The diet envisages the complete exclusion of certain cereals and their derivatives and if not carefully monitored is often lacking in group B vitamins, calcium, vitamin D, iron, zinc, magnesium and fibre. In addition, the malabsorption resulting from atrophy of the intestinal villi causes weight loss, constipation or diarrhoea, iron deficiency, vitamin B12 and folate deficiency anaemia in coeliacs. Finally, the elimination of cereals from the diet of coeliac patients determines a reduced fibre intake.

The food products currently developed to meet the dietary needs of coeliac patients are exclusively characterised by the use of naturally gluten-free ingredients or ingredients that are de-glutenised by means of chemical treatment processes.

Therefore, while in the strict sense preventing the immune reaction is triggered by gluten, these products do not however adequately meet the nutritional needs of coeliac patients as they do not in fact provide functional supplementation to the states of deficiency that affect coeliac patients. This is all the more true and important for those patients, for whom there was late diagnosis of the disease, and in whom there is observed an especially severe state of malnutrition due to prolonged lesion and atrophy or the intestinal villi.

Furthermore, coeliac patients have a tendency to develop veno-occlusive and arterial occlusive diseases, a tendency that is accentuated by the general state of malabsorption that sets coeliac patients apart. In particular, the group B vitamin deficit (and consequently an increase in plasma homocysteine) is a commonly found condition. It should furthermore be added that regardless of the vitamin B deficit, coeliac disease is in itself a fundamental determining factor of homocysteine values, which is therefore also considered to be a very sensitive indicator of states of vitamin deficiency.

Lastly, coeliac patients present a more or less marked incapacity of the stomach to secrete a substance called “intrinsic factor”, necessary for the intestinal absorption of vitamin B12, which, in collaboration with the folates, activated the haemopoiesis process. The lack of these vitamins determines more or less severe forms of anaemia in coeliac patients.

There is therefore a strong need to make available to coeliac patients food products that are not only gluten-free but that also, above all, integrate and supplement the states of deficiency that are typical of the gluten-free diets.

The object of the present invention is therefore to identify food formulations that overcome the aforementioned drawbacks.

### SUMMARY OF THE INVENTION

The above indicated object has been achieved by a gluten-free food composition consisting of at least one derivative of Cannabis Sativa, at least one derivative of 5-methyltetrahydrofolic acid, and suitable food ingredients.

It has in fact been surprisingly observed that food products that are perfectly suitable for the diet of gluten-intolerant patients can be prepared with the combined properties of the derivatives of Cannabis sativa, which are rich in essential fatty acids, amino acids, vitamin E and of the derivatives of 5-methyltetrahydrofolic acid, thus not only allowing the absorption of substances not tolerated by the bodies of such patients to be prevented, but also allowing their diet to be supplemented with elements that assist the re-establishment of the correct functioning of the body.

Thus, in another aspect, the present invention concerns food products comprising said composition, in the form of sweet and savoury baked products, preparations for sweet and savoury products, cornflakes, bread, pasta, pizza, croissants, cake, pie, chocolate cream, biscuits, crackers, muesli, diet bars, candy, chocolate or vegetal milk.

The characteristics and the advantages of the present invention will be clear from the following detailed description and working examples provided for illustrative and non-limiting purposes.

### DETAILED DESCRIPTION OF THE INVENTION

The invention thus has as object a gluten-free food composition consisting of at least one derivative of Cannabis Sativa, at least one derivative of 5-methyltetrahydrofolic acid and suitable food ingredients.

For the purposes of the present invention, the expression “derivative of Cannabis Sativa” shall mean virgin Cannabis Sativa oil or a derivative thereof, wholemeal or refined Cannabis Sativa flour, edestin or mixtures thereof.

For the purposes of the present invention, the expression “derivative of virgin Cannabis Sativa oil” shall mean C₁-C₄ alkyl ester of polyunsaturated fatty acid present in the Cannabis Sativa oil. Said derivative is preferably a mixture of C₁-C₄ alkyl esters of polyunsaturated fatty acids present in Cannabis Sativa oil. Said derivative is more preferably a mixture of alkyl esters of polyunsaturated fatty acids present in Cannabis Sativa oil.

Wholemeal flour, refined flour and virgin oil obtained from Cannabis sativa seed present a nutritional profile that is characterised by a high bioavailability of is essential fatty acids (linoleic acid, α-linolenic acid, γ-linolenic acid, stearidonic acid).

For the purposes of the present invention, the expression “derivative of 5-methyltetrahydrofolic acid” shall mean an alkaline or alkaline-earth salt of said acid or a precursor of said acid, such as 5-formyltetrahydrohydrofolic acid, 5-methyltetrahydro-pteroyltriglutamic acid, 10-formyltetrahydrofolic acid, or relevant isomers (6R)-, or (6S)-.

Coeliac disease is characterised by chronic inflammation of the mucosa of the small intestine with an inefficient epithelial transport. The fatty acid composition of the intestinal membranes is important for epithelial function and an insufficiency thereof can contribute to the pathophysiology of the disease.

It has in fact been observed that the proportions of Arachidonic acid (20:4 n-6) and the polyunsaturated fatty acids significantly decrease in subjects with active coeliac disease. The serum levels of these fatty acids increase during remission, albeit remaining significantly lower than those of people not subject to the disease. An increase in the levels of unsaturated and monounsaturated fatty acids is associated with this decrease.

More interesting is noting how significant differences compared to healthy subjects can be observed in the intestinal mucosa affected by coeliac disease. In particular, the level of linoleic acid (18:02 n-6) is significantly reduced, while the level of eicosatrienoic acid (20:03 n-9) increases, thus indicating a lack of essential fatty acids.

It is indeed the supplementation, in the ratio of 3:1, of essential omega-6 (linoleic acid and γ-linolenic acid) fatty acids and essential omega-3 α-linolenic and stearidonic acid) contained in the food composition of the invention, that intervenes in re-establishing the correct presence thereof in the intestinal mucosa, thus reducing the inflammatory effect determined by insufficiency thereof.

Said at least one derivative of hemp is therefore preferably wholemeal or refined Cannabis Sativa flour, virgin Cannabis Sativa oil or a mixture thereof.

These benefits are however mild in extent and duration if it is not possible to intervene in the functional repair of the epithelial cells of the intestinal tract. At this level, it is in fact necessary to intervene to reactivate the physiological cell is regeneration mechanisms and rebalancing mechanisms for blood homocysteine levels even when the correct trophy of the villi has yet to be restored.

By combining, in food products, wholemeal flour, refined flour and/or virgin oil obtained from Cannabis Sativa seed with folates it is possible to also provide, in addition to the advantages in terms of reduced inflammation, an adequate response to the three-fold requirement to stimulate the growth and reproduction process of the intestinal epithelial cells, to reduce homocysteine and to form haem. Folates are vitamins that are necessary for the synthesis, the repair and functioning of DNA and RNA. They are therefore fundamental for the production and maintenance of new cells, particularly in periods of a necessary rapid growth. One of the two principal metabolic homocysteine pathways that maintain its intra-cellular levels within a certain range is the so-called remethylation pathway. Homocysteine is converted into methionine through the use of three enzymes: methionine-synthetase, methylenetetrahydrofolate red uctase and betaine synthetase. This metabolic process comes into play when there are low concentrations of homocysteine and of methionine. In the second case, the enzyme used is the cystathionine-β-synthetase enzyme and the end result of the degradation is the amino acid, cysteine. Certain B group vitamins are involved in these processes: vitamin B9 (folates), betaine (trimethylglycine), vitamin B12 (cyanocobalamin), vitamin B6 (pyridoxine) and vitamin B3 (riboflavin). When the metabolic pathways reach saturation, the intracellular homocysteine enters into circulation and there is an increase in the plasma levels of this amino acid; at this point the homocysteine can bind to the plasma proteins or can be eliminated by renal route.

Folates are active in cell division and carry out their fundamental role as carbon carriers in the formation of haem, the protein containing iron that is found in haemoglobin, necessary for the formation of red blood cells.

Folates present in foods are generally somewhat unstable and recent studies believe that their bioavailability is around 80% that of folic acid, the synthetic, oxidised, chemically stable form of the vitamin, which is used for supplementation and fortification. Folic acid supplementation in coeliac patients can however give rise to masking of the vitamin B12 deficiency.

Supplementation with said at least one derivative of 5-methyltetrahydrofolic acid has resulted in a surprisingly advantageous alternative to folic acid, in that it allows the folate status to be raised without masking the vitamin B12 deficiency.

In fact, said at least one derivative of 5-methyltetrahydrofolic acid has proved to be safe and efficient in raising folate plasma concentrations in red blood cells and its bioavailability has proved to be even greater that than that of folic acid.

Said at least one derivative of 5-methyltetrahydrofolic acid is preferably an alkaline or alkaline-earth salt of 5-methyltetrahydrofolic acid; more preferably it is a salt of Na, K, Li, Ca, Ba, Mg or a mixture thereof.

According to one preferred embodiment, said at least one salt of 5-methyltetrahydrofolic acid is a calcium salt of [6S]-5-methyltetrahydrofolic acid ([6S]-5 MTHF).

Preferably, in the food composition of the invention, said at least one derivative of Cannabis Sativa is in an amount of 0.05-40 wt %, and said at least one derivative of 5-methyltetrahydrofolic acid is in an amount up to 0.001 wt % on the total weight of the composition.

More preferably, in the food composition of the invention, said at least one derivative of Cannabis Sativa is in an amount of 0.5-30 wt %, and said at least one derivative of 5-methyltetrahydrofolic acid is in an amount up to 0.0006 wt % on the total weight of the composition.

According to a preferred embodiment, said at least one derivative of Cannabis Sativa is in an amount of 1-25 wt %, and said at least one derivative of 5-methyltetrahydrofolic acid is in an amount up to 0.0004 wt % on the total weight of the composition.

Among the suitable food ingredients, the food composition of the invention can contain at least one other type of gluten-free flour, i.e. corn flour, rice flour, millet flour, buckwheat flour, quinoa flour, potato starch, amaranth flour, tapioca flour, cassava flour, chickpea flour, soy flour, pea flour, lupin flour or a mixture thereof.

Preferably, said at least one other type of gluten-free flour is in an amount of 10-60 wt % on the total weight of the composition.

For the purposes of the present invention, the possibility of obtaining food products that differ greatly one from the other is of fundamental importance with a view to providing a diet that is strictly gluten-free, but at the same time pleasantly diverse and appetising. The preparation of sweet and savoury baked products, preparations for sweet and savoury products, cornflakes, bread, pasta, pizza, croissants, focaccia, cakes, crème de cacao, biscuits, crackers, muesli, diet bars, candy, chocolate or vegetal milk is in fact possible.

Example embodiments of the present invention are provided by way of illustration below.

### EXAMPLE 1

### Preparation of Bread Comprising the Composition of the Invention

Bread having the following ingredient composition, expressed in % wt, was prepared:

| Ingredients | |
|---|---|
| Corn flour | 43.75% |
| Cannabis Sativa seed flour | 20% |
| Water | 31.99975% |
| Inulin | 3.5% |
| E-471(*) | 0.75% |
| Calcium [6S]-5-methyltetrahydrofolate | 0.00025% |

(*)Emulsifying agent comprising mono and diglycerides of fatty acids

### EXAMPLE 2

### Preparation of Pasta Comprising the Composition of the Invention

Pasta having the following ingredient composition, expressed in % wt, was prepared:

| Ingredients | |
|---|---|
| Corn flour | 45% |
| Cannabis Sativa seed flour | 12.25% |
| Buckwheat flour | 10% |
| Water | 31.68% |
| E-471 | 0.95% |
| Curcumin | 0.1196% |
| Calcium [6S]-5-methyltetrahydrofolate | 0.00040% |

### EXAMPLE 3

### Preparation of Pasta Comprising the Composition of the Invention

Pasta having the following ingredient composition, expressed in % wt, was prepared:

| Ingredients | |
|---|---|
| Corn flour | 28% |
| Rice flour | 24% |
| Cannabis Sativa seed flour | 12% |
| E-472(**) | 0.9496% |
| Inulin | 1.05% |
| Water | 34% |
| Calcium [6S]-5-methyltetrahydrofolate | 0.00040% |

(**)Emulsifying agent comprising monoacetylethyltartaric and diocetyltartaric esters of the mono and diglycerides of fatty acids.

### EXAMPLE 4

### Preparation of Crackers Comprising the Composition of the Invention

Crackers having the following ingredient composition, expressed in % wt, were prepared:

| Ingredients | |
|---|---|
| Corn flour | 34.6% |
| Cannabis Sativa seed flour | 16% |
| Cannabis Sativa seed oil | 6% |
| Water | 32% |
| Sugar | 0.1% |
| Salt | 0.05% |
| Guar gum | 2.5% |
| E-472 | 0.8% |
| Dried thyme | 1% |
| Brewers' yeast | 3.5% |
| Psyllium seed fibre | 3.2% |
| Sodium pyrophosphate | 0.2496% |
| Calcium [6S]-5-methyltetrahydrofolate | 0.00040% |

### EXAMPLE 5

### Preparation of Crackers Comprising the Composition of the Invention

Crackers having the following ingredient composition, expressed in % wt, were prepared:

| Ingredients | |
|---|---|
| Corn flour | 38.5% |
| Cannabis Sativa seed flour | 12% |
| Cannabis Sativa seed oil | 4.5% |
| Water | 35% |
| Sugar | 0.15% |
| Salt | 0.05% |
| Xanthan gum | 0.6% |
| Hydroxylpropyl-methylcellulose | 1.5% |
| E-471 | 0.24975% |
| Ascorbic acid | 0.25% |
| Sodium propionate | 0.1% |
| Dried olives | 2% |
| Dried capers | 0.8% |
| Dried oregano | 1.5% |
| Cornflour | 2.8% |
| Magnesium [6S]-5-methyltetrahydrofolate | 0.00025% |

### EXAMPLE 6

### Preparation of Biscuits Comprising the Composition of the Invention

Biscuits having the following ingredient composition, expressed in % wt, were prepared:

| Ingredients | |
|---|---|
| Rice flour | 28% |
| Cannabis Sativa seed flour | 12% |
| Cannabis Sativa seed oil | 3.58% |
| Water | 25% |
| Sugar | 2.5% |
| Xanthan gum | 1.2% |
| Hydroxylpropyl-methylcellulose | 2% |
| Guar gum | 0.25% |
| E-472 | 0.36% |
| Ascorbic acid | 0.05975% |
| Calcium carbonate | 0.1% |
| Cornflour | 2.5% |
| Brewers' yeast | 1.6% |
| Inulin | 3.5% |
| Sodium bicarbonate | 0.1% |
| Chocolate | 3.5% |
| Eggs | 4.25% |
| Butter | 3.5% |
| Powdered milk | 6% |
| Calcium [6S]-5-methyltetrahydrofolate | 0.00025% |

### EXAMPLE 7

### Preparation of Biscuits Comprising the Composition of the Invention

Biscuits having the following ingredient composition, expressed in wt %, were prepared:

| Ingredients | |
|---|---|
| Rice flour | 15% |
| Cannabis Sativa seed flour | 12.98% |
| Millet flour | 26% |
| Cannabis Sativa seed oil | 3% |
| Water | 25% |
| Sugar | 2% |
| Salt | 0.2% |
| Xanthan gum | 0.05% |
| Hydroxylpropyl-methylcellulose | 0.15% |
| Guar gum | 0.25% |
| E-472 | 0.02% |
| Ascorbic acid | 0.01% |
| Sodium propionate | 0.01% |
| Potato starch | 2% |
| Brewers' yeast | 0.75% |
| Sodium bicarbonate | 0.01975% |
| Vanillin | 0.01% |
| Eggs | 3% |
| Sodium pyrophosphate | 0.25% |
| Butter | 4.3% |
| Powdered milk | 5% |
| Potassium [6S]-5-methyltetrahydrofolate | 0.00025% |

### EXAMPLE 8

### Preparation of Chocolate Comprising the Composition of the Invention

Chocolate having the following ingredient composition, expressed in wt %, was prepared:

| Ingredients | |
|---|---|
| Cocoa paste | 82% |
| Cocoa butter | 10% |
| Dehusked hemp seed | 5% |
| Cannabis Sativa seed oil | 1.99% |
| Sugar | 1% |
| Dried chilli | 0.0096% |
| [6S]-5-methyltetrahydrofolate | 0.00040% |

The advantages achieved by means of the food composition of the invention are clear from the detailed description and from the above listed Examples. Said food io composition allows food products that differ greatly one from the other to be obtained with a view to providing a diet that is strictly gluten-free but at the same time pleasantly diverse and appetising. It fact, food products that are perfectly suitable for the diet of gluten-intolerant patients can be prepared with the combined properties of the derivatives of Cannabis sativa, which are rich in essential fatty acids, amino acids, vitamin E and of the 5-methyltetrahydrofolic acid salts, thus not only allowing the absorption of substances not tolerated by the bodies of such patients to be prevented, but also allowing their diet to be supplemented with elements that assist the re-establishment of the correct functioning of the body.

1. Gluten-free food composition consisting of at least one derivative of Cannabis Sativa, at least one derivative of 5-methyltetrahydrofolic acid, and suitable food ingredients.

2. The composition of claim 1, wherein said at least one derivative of Cannabis Sativa is wholemeal or refined Cannabis Sativa flour, virgin Cannabis Sativa oil or a mixture thereof.

3. The composition of claim 1 or 2, wherein said at least one derivative of 5-methyltetrahydrofolic acid is salt of Na, K, Li, Ca, Ba, Mg, or a mixture thereof.

4. The composition of claim 3, wherein said salt of 5-methyltetrahydrofolic acid is [6S]-5-methyltetrahydrofolate calcium salt.

5. The composition of claim 1, wherein said at least one derivative of Cannabis Sativa is in an amount of 0.05-40 wt % and said at least one derivative of 5-methyltetrahydrofolic acid is in an amount of up to 0.001 wt % on the total weight of the composition.

6. The composition of claim 5, wherein said at least one derivative of Cannabis Sativa is in an amount of 0.5-30 wt % and said at least one derivative of 5-methyltetrahydrofolic acid is in an amount of up to 0.0006 wt % on the total weight of the composition.

7. The composition of claim 6, wherein said at least one derivative of Cannabis Sativa is in an amount of 1-25 wt % and said at least one derivative of 5-methyltetrahydrofolic acid is in an amount of up to 0.0004 wt % on total weight of the composition.

8. The composition of claim 1, wherein said suitable food ingredients comprise one other type of gluten-free flour selected from corn flour, rice flour, millet flour, buckwheat flour, quinoa flour, potato starch, amaranth flour, tapioca flour, cassava flour, chickpea flour, soy flour, pea flour, lupin flour, or a mixture thereof.

9. The composition of claim 8, wherein said one other type of gluten-free flour is in an amount of 10-60 wt % on the total weight of the composition.

10. Food product comprising the composition claim 1, in the form of a sweet or savoury baked product, a preparation for sweet and savoury products, cornflakes, bread, pasta, pizza, croissant, cake, pie, chocolate cream, biscuit, crackers, muesli, diet bar, candy, chocolate, or vegetal milk.
